(19) United States
(12) Patent Application Publication
(10) Pub. No.: US 2005/0014830 A1
(43) Pub. Date: Jan. 20, 2005

(54) Substances for breaking down conformation of microbes

(76) Inventors: Shih Min Lin, Taipei (TW); Chih Kung Lee, Taipei (TW); Shih Yuan Lee, Taipei (TW); Shu Fang Chu, I lan City (TW); Wen Hsin Hsiao, Lung tan Shiang (TW)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/860,648

(22) Filed: Jun. 4, 2004

(30) Foreign Application Priority Data
Jul. 17, 2003 (TW) 92119530

(51) Int. Cl. A61K 31/20

(52) U.S. Cl. 514/558

(57) ABSTRACT

A category of substances for inflicting in a biomolecular mechanism the conformation breakdown of microbes into biological components is disclosed. A substance of the category has a chemical composition of hydrophobic nature having bonded to at least one structural point of its composition a hydrophilic functional group. A multiplicity of molecules of the substance inflicts the microbe breakdown by being adsorbed to the microbial surface of the target microbe and assembled into aggregates. The substance molecules' aggregates at close proximity to the microbial surface exert adsorptive interaction forces to nearby ones of the microbe biological components. The forces exerted are significant enough to compete with the absorptive interaction forces among the biological components and thereby disrupts the conformation, breaks out of structural binding equilibrium among the biological components, and results in the breakdown and denaturation of the microbe.

### BACKGROUND OF THE INVENTION

1. Field of the Invention

This invention relates in general to substances for inflicting conformation breakdown of microbes. In particular, this invention relates to an innovative category of chemical substances that effectively break down microbes for disabling and disinfecting viruses and bacteria.

2. Technical Background

Microbes such as viruses and bacteria are microorganisms responsible for many diseases, many of which are fatal to human if infected. Various medical treatments including vaccination and drug cures are available for human in the defense against known microbes. However, for known or unknown microbes alike for which no known defense or cure are available are fierce enough to inflict high mortality and are capable of efficient infection schemes such as airborne transmission, the first and only effective means for protection is disinfection.

Disinfection of microbes can be achieved physically and chemically. Physical disinfection schemes include heating, drying, freezing, radioactive irradiation and filtration, etc. These physical disinfection schemes are relatively constrained in terms of factors such as characteristics of environments in which to perform such processing. Chemical disinfection schemes are thus more practically applicable for defense against microbes.

Materials and substances generally used as chemical disinfectants include acid, alkaline, alcohol, carbolic acid, formaldehyde, surfactants, halogen, oxidants, heavy metals and dyes etc. In principle, they achieve microorganism disinfection chemically via one or more of four of the following effects: damages to cell membrane, destruction of cellular transportation, microbial protein denaturation, and enzyme reactivity and/or receptor affinity suppression.

Effectiveness of chemical disinfectants is varied. In principle, the more effective is a chemical disinfectant, the more hazardous it is likely to human. Selection of disinfectants depends on factors including the type of the target microbe, characteristics of the site to apply disinfection, and disease prevention requirements, among others.

In general, an ideal chemical disinfectant should qualify the following characteristics: effectiveness against a broad variety of microbes; negligible susceptibility to organic compounds, superior microbial surface penetration capability; non-corrosive, non-toxic and non-irritative to human; chemical stability with accelerated disinfecting effectiveness; high water solubility; sustained adherence to the surface of the disinfected object for sustained microbe suppression capability; and reasonable cost for mass production.

### SUMMARY OF THE INVENTION

The present invention provides an innovative category of substances for breaking down the conformation of a microbe into biological components, said substance having a chemical composition of hydrophobic nature having bonded to at least one structural point thereof a hydrophilic functional group, wherein a multiplicity of molecules of said substance being adsorbed by said hydrophilicity to the microbial surface of said microbe and assembled gradually into aggregates, said aggregates at close proximity of said microbial surface exerting adsorptive interaction forces to nearby ones of said microbe biological components competing with the absorptive interaction forces among said nearby biological components, and said competition disrupting said conformation by breaking out of structural binding equilibrium among said biological components and thereby breaking down and denaturing said microbe.

### BRIEF DESCRIPTION OF THE DRAWINGS

FIG. 1 schematically illustrates the molecular structural configuration of a preferred embodiment of the chemical composition of the innovative substance;

FIG. 2 schematically illustrates the molecular structural configuration of another preferred embodiment of the chemical composition of the innovative substance having additional functional group;

FIG. 3 illustrates the introduction of the innovative substance molecules and the target microbe in the same solution allowing the initiation of the microbe-breaking mechanism;

FIG. 4 illustrates the approaching of the innovative substance molecules toward a microbe and the accompanying bulk aggregation and the adsorption to the microbial surface for interaction with the biological components nearby;

FIG. 5 illustrates the competition of accumulated forces of multiplied physical adsorption with forces of physical absorption between biological components on the microbial surface of the microbe;

FIG. 6 illustrates the severely interfered microbe conformation by the presence of the adsorptive interaction forces significantly competing with physical absorptions between biological components of target microbe;

FIG. 7 illustrates a target microbe of disrupted and broken 3-D structural binding equilibrium among its biological components that is effectively denatured;

FIG. 8 shows a chemical reaction in which the hydrocarbon long chain of the molecule of the inventive substance is linked to the surface of a polystyrene-based plastic material;

FIGS. 9A˜9D respectively are atomic force microscope (AFM) pictures of a SARS virion taken at different stages of treatment by the innovative 8-hydroxyoctanoic acid organic substance of the present invention;

FIGS. 10A and 10B respectively are the AFM height images of an enterovirus virion before and after the treatment by the inventive substance 8-hydroxyoctanoic acid; and

FIGS. 11A and 11B respectively are the AFM pictures of a bacterium of the Serratia species taken before and after the treatment by the innovative 8-hydroxyoctanoic acid organic substance of the present invention.

### DETAILED DESCRIPTION OF THE PREFERRED EMBODIMENTS

An understanding to the infectiousness of viruses and bacteria in terms of their micro-organic structures and the biochemical/biophysical interactions with the target hosts they infect is key to the development of an effective microorganism disinfectant. In the case of viruses responsible for more than half of known diseases to human beings, the virion of a contagious pathogen in all is a relatively simple microscopic organism. Essentially, a virus can be as simple as a set of genetic information-carrying nucleic acids enclosed in a protective protein capsid. Some viruses have an additional envelope surrounding the capsid, and still others have protruding spikes.

A simple virus can not reproduce by itself. A host must be available to the virus, and the nucleic acid and protein reproducing mechanism of the host cell is used by the virus to duplicate itself. Not all cells of an infected host, however, are available to a virus to infect and hijack. Only those host cells with biomolecular affinity to the virus, much like the exact paired matching of the key and its corresponding keyhole, are of use to the virus for its reproduction.

With a specific biomolecular matching system, an approaching virus can selectively inflict its infection against specific target host cells. The identifying mechanism of a virus is on the microbial surface of its capsid or envelope that can be matched to specific compatible receptors of the victim host cell. Disintegration of this victim-identifying biomolecular structure thus implies the denaturation, or, the loss of infectiousness, of a virus.

Virus capsid and envelope are, in principle, a layer of protein such as lipoprotein or glycoprotein which, when broken down or denatured, has the infectiousness to its target host cells denatured. However, for some viruses and bacteria without capsid or envelope, enterovirus for example, internal microbial components have to be digested or denatured if they were to be disinfected.

In a proposed innovative biophysical mechanism of mutual interactions between matters at the molecular level, the present invention discloses how the use of a newly-sorted category of chemical substances can achieve the conformation breakdown of microorganisms by such envelope and/or capsid denaturation.

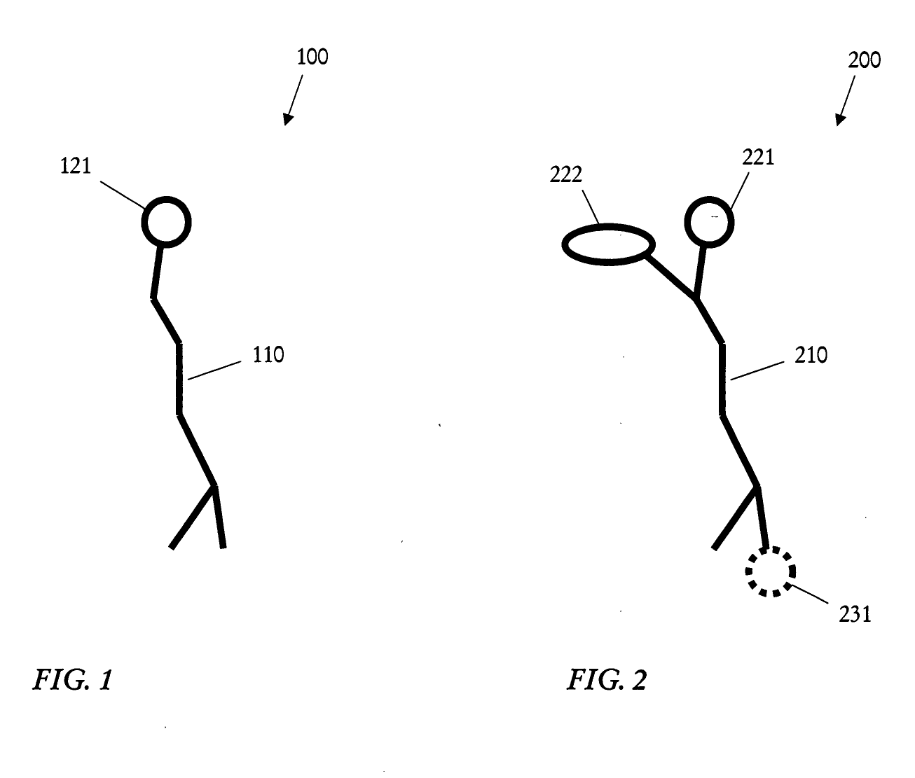

The new category of substances is the result of a set of biochemical and biophysical design thinking derived from this microorganism breakdown mechanism. It has been shown that the new category of organic chemical substances thus developed is more than effective in denaturing the conformation of viruses. The substances are also effective in collapsing, at least partially, the conformation of bacteria. FIGS. 1 and 2 schematically illustrate the structural configuration of the preferred embodiments of the chemical composition of the innovative substance.

As is illustrated in FIG. 1, a preferred embodiment of the substance of the present invention has an elongated chemical composition. An individual molecule 100 of the chemical substances in this category is characterized by the chemical composition of a hydrophobic long chain 110 of hydrocarbon, with at least one hydrophilic functional group 121 on the long chain, preferably at an end of the molecular composition thereof.

Within the scope of the present invention, a hydrocarbon of a long-chain composition may also include those with single- (C—C), double- (C═C)) and/or triple-bond (C≡C) carbon-carbon structures. Long chains which are comprised partially of formation bonds of carbon with heteroatom such as carbon-oxygen (C—O) and carbon-nitrogen single-bonds (C—N) are also considered to be within the scope of the present invention.

FIG. 2 schematically illustrates, at the molecular level, the structural configuration of another embodiment of the chemical composition of the innovative substance having additional functional groups than that of FIG. 1. An additional hydrophilic functional group 222, schematically shown to be a different chemical group than 221, is also bonded to the same end of the elongated chain 210 of the substance molecule 200. Further, at least one functional group 231 of desired chemical characteristics can optionally be bonded to the long chain 210 at the end opposite to that having hydrophilic functional group or groups.

### Biophysical Interactions in a Microbe-Breakdown Mechanism

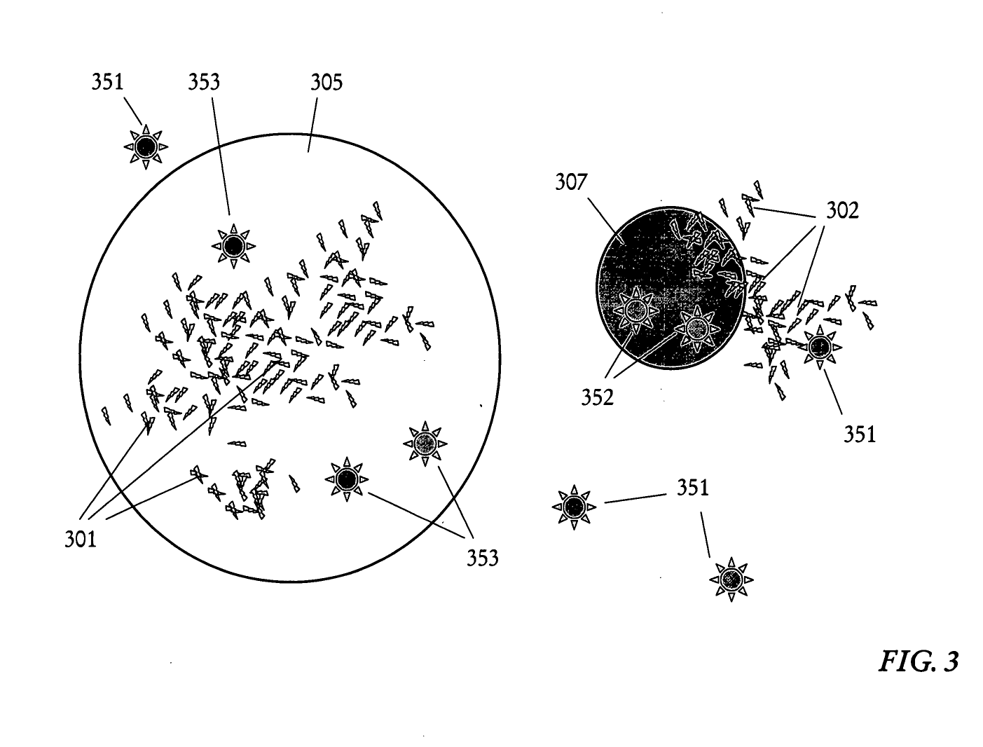

The disclosed innovative category of substances inflicts the conformation breakdown of microbes into biological residual components that can be described in a biomolecular interaction mechanism. A substance of the category has a chemical composition of an elongated chain of hydrophobic nature having bonded to at least one structural point of its composition a hydrophilic functional group. FIGS. 3 to 7 schematically illustrate in a time sequence the microbe breakdown mechanism inflicted by the innovative substance of the present invention.

Note that in the context of the description of the present invention, the terms adsorb, adsorption and adsorptive forces refer to the interactions between molecular components of a microorganism and the foreign treatment matters of the substances. By contrast, the terms absorb, absorption and absorptive force refer to the molecular interactions between microbial components of the microorganism itself.

FIG. 3 illustrates the introduction of the innovative substance molecules and the target microbe in the same environment, usually a solution, allowing the initiation of the microbe-breaking mechanism. The drawing exemplifies several scenarios the inventive substance can be used for disinfection of disease-inflicting microorganisms. Consider the case in which coronavirus such as the severe acute respiratory syndrome (SARS) virus is present in an environment that requires disinfection. Some SARS virions 351 may litter around a human environment such as those settled on the surface of furniture pieces or appliances. Still others 352 may be carried in body fluid droplets 307 expelled by a contagious patient that may be airborne or may have landed on some surface in the environment.

To facilitate disinfection of these dangerous environmental microorganisms, microbe-breaking substances of the present invention can be deployed into the human environment in several ways. For example, molecules 301 of the inventive substance can be contained in a solution and sprayed as disinfection droplets 305 to land on the human access surfaces. They can also be applied in cleaning solutions via wet-wiping as well. Or, molecules 302 of the inventive substance can be engineered, as will be described hereinafter, to securely affix to surfaces of appliances or furniture pieces for prolonged disinfection efficacy against environmental disease-inflicting microbes.

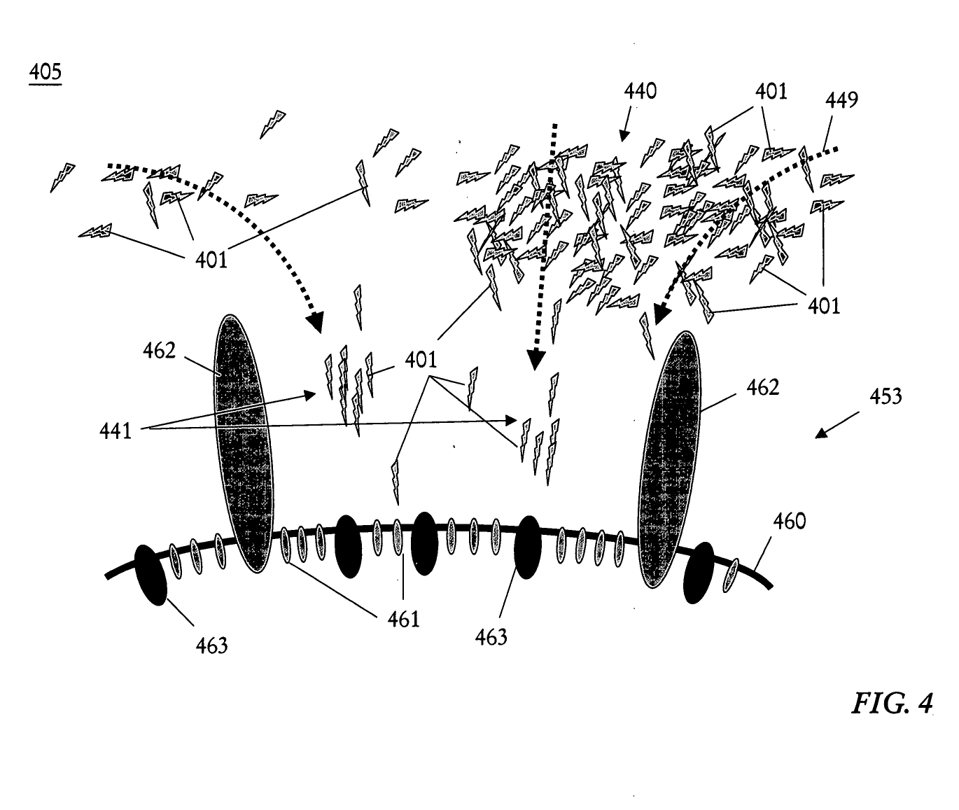

In FIG. 4, the innovative substance molecules 401 are shown to be approaching toward a target microbe 453. This happens once both the substance molecules and the virions are brought into the same solution such as when the disinfection droplet 305 in FIG. 3 lands on spot of the environmental surface where some SARS virions 353 are present.

In the aqueous solution generally denoted by reference numeral 405 containing a typical concentration of the inventive substance, molecules of the substance are evenly distributed and exhibit no bulk order of their molecular axial orientations. This is exemplified schematically in the drawing by the group of the bulk 440 of substance molecules suspended in the solution 405, sufficiently far away from the microbial surface 460 of the target virion 453.

This equilibrium, however, is eventually disturbed via the presence of foreign matters in the same solution due to the introduction of the target microorganisms to be disinfected. Hydrophilic functional group (or groups) at the end of the hydrophobic long chain of the chemical composition of each substance molecule induces molecular affinity to the microbial surface of the target virions.

The hydrophilicity-induced mutual affinity between the substance molecules' hydrophilic functional groups and the microbial surface 460 of a target virion 453 gradually aligns the orientation of the substance molecules 401 as they are adsorbed toward the target virion. Observed, molecules 401 of the inventive substance in the treatment solution 405 closer to the microbial surface 460 of the virion 453 appear to be adsorbed first by the biological components 461 and 462 at the surface 460. Depending on the nature of the virus, components such as membrane proteins 461, envelope proteins 463 and spikes 462 in the case of a coronavirus such as SARS virus provide the close-proximity bioaffinity to the substance molecules, gradually resulting into the initial aggregates of substance molecules such as identified in the drawing as aggregates 441. The bulky-growing aggregations due to the adsorption of the substance molecules to the microbial surface gradually inflict their amplified interaction onto the biological microbial components of the target microbe nearby.

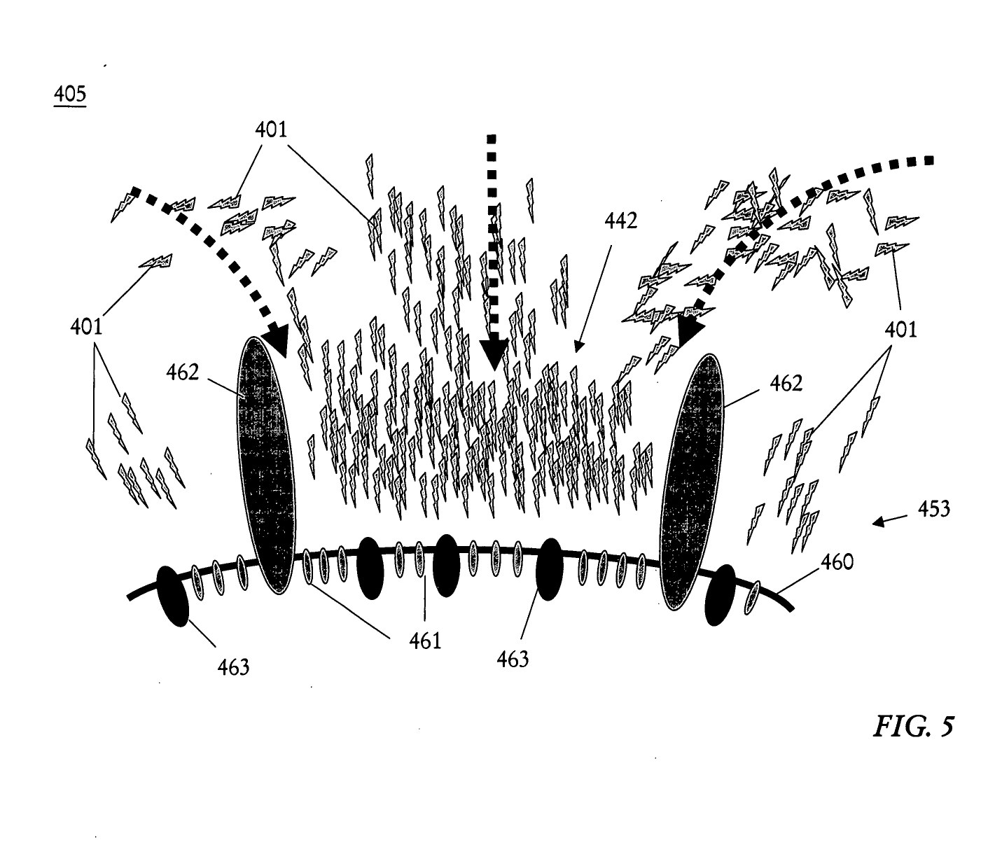

In FIG. 5, aggregates such as that generally identified as 442 that are sufficiently bulky give rise to the competition between substance molecules' accumulated forces of multiplied physical adsorption and the forces of physical absorption among the biological components proximate to the microbial surface of the microbe. These molecular interaction force competitions eventually become significant enough to distort and twist the conformational structure of the composition of the reacted virion.

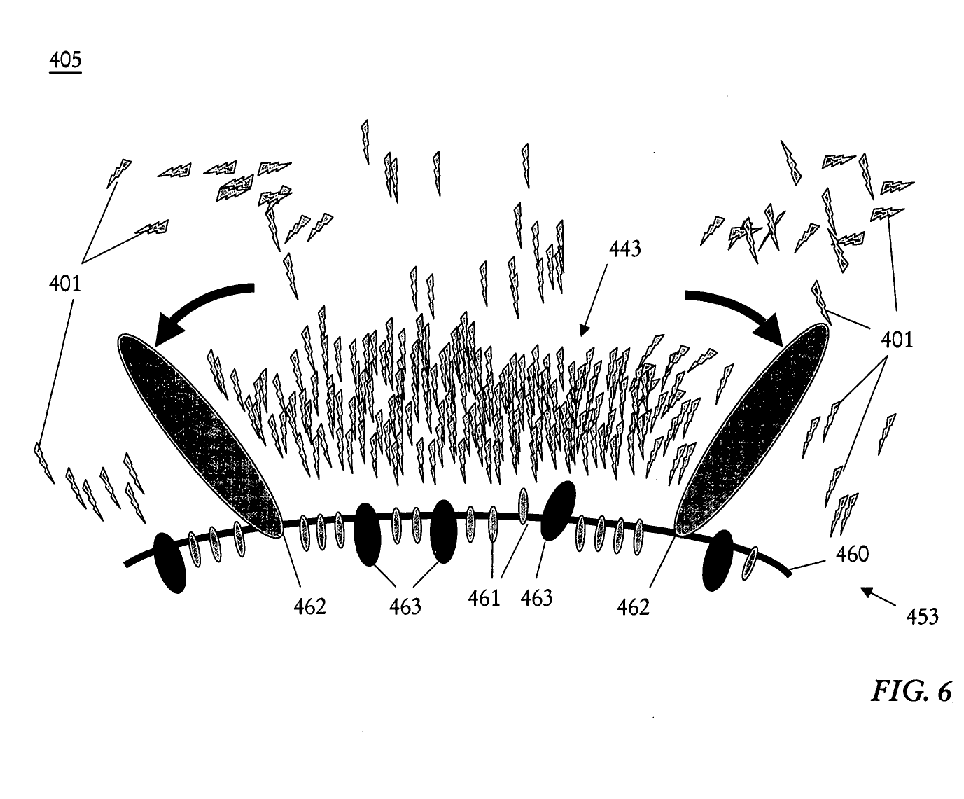

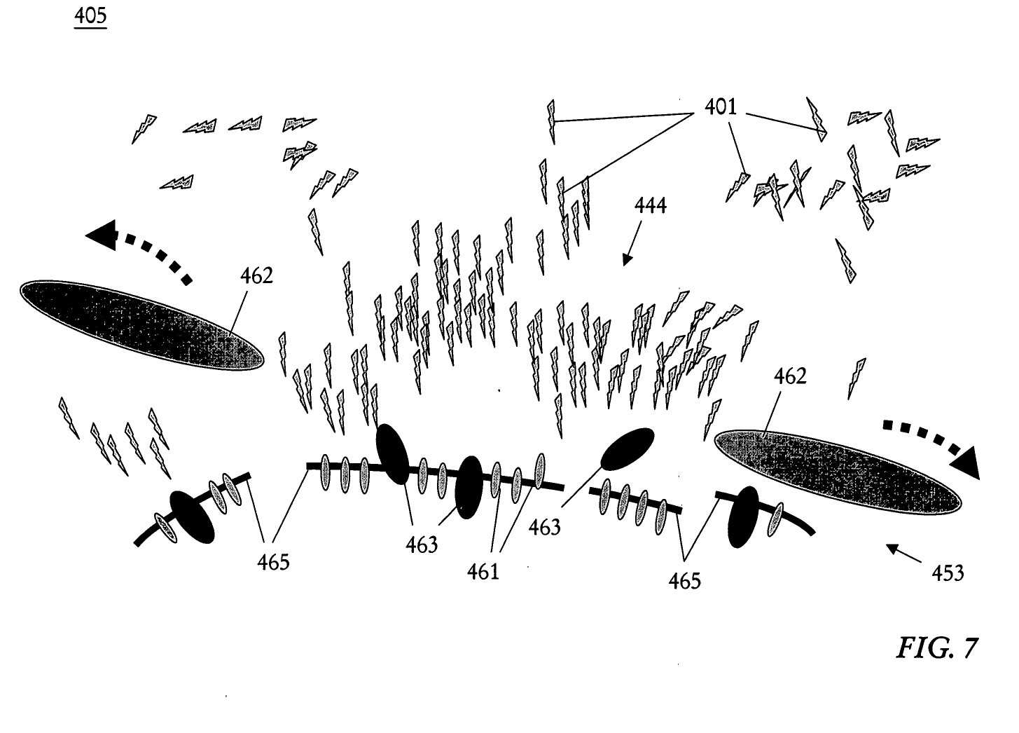

FIG. 6 illustrates the severely interfered microbe conformation by the presence of the adsorptive interaction forces significantly competing with physical absorptions between biological components of target microbe. The main aggregation 443 grown from 442 in the previous time stage of FIG. 5 has finally accumulated to a level sufficient to exert disruptive force on the microbial conformation system that breaks down the target virion as illustrated in FIG. 7. In the schematic drawing, the target microbe 453, with disrupted and broken 3-D structural binding equilibrium among its biological components, becomes effectively denatured.

In the process, competing adsorptive and absorptive interactions mixed among aggregates of substance molecules and the virion microbial biological components disrupt equilibrium in the microbe's structural component binding system. The result is the breakdown of the microbial 3-D conformation, leaving behind littered biological components 461 and 462 and residues of the envelope 465 of the disinfected virion.

FIGS. 3 to 7 thus describe a biophysical mechanism showing that a sufficient abundance of the innovative chemical substance molecules can be used to denature the structural conformation of a microbe. Initially, a multiplicity of molecules of the substance inflicts the microbe breakdown by being adsorbed to the microbial surface of the microbe and gradually assembled in aggregates in the process. Substance molecules eventually aggregated at close proximity of the microbial surface exert adsorptive interaction forces to nearby ones of the microbe biological components. The exerted interaction gradually constitutes interference forces significant enough to compete with the absorptive interaction forces among the biological components. As the aggregation becomes significant enough, the substance molecules thereby disrupts the conformation of the target microbe, breaks out of structural binding equilibrium among the biological components, and results in the breakdown of the microbe.

### Examples of the Microbe Disinfecting Substance

In accordance with the teaching of the present invention, a special category of chemical substances are specifically designed and developed based on the thoughts of an innovative microbe disabling and denaturing mechanism. The proposed interaction mechanism offers an interaction model that seeks to describe and explain, at the biomolecular level, the physical interactions between matters of the inventive substances themselves and that of the target microbes. Chemical substances in this category are characterized by the chemical composition of a hydrophobic long chain of hydrocarbon, with at least one hydrophilic functional group at an end of the long chain, and optionally at least another group of desired chemical characteristics at the other end, or at another branched end of the main chain. This optional and additional functional group can be either hydrophobic or hydrophilic according to the requirement of application design.

The hydrophilic functional group(s) at one end of the substance chemical composition is vital to the substance's ability to disrupt and disintegrate the three-dimensional conformation of the microorganism. On the other hand, the optional functional group or groups at the opposite end of the long chain of the substance serve to provide additional usefulness such as strong physical linkage to materials such as textiles, plastics and metals, whose prolonged disinfecting capability against microorganisms is desirable in daily life.

Such chemical substances are capable of digesting and denaturing microorganisms including viruses and bacteria. For example, for the desirable functionality of microbe disinfection, the innovative chemical substances of the present invention are capable of and have demonstrated the damage to the three-dimensional conformation of the relatively more complex bacteria. When it comes to viruses, the substances are capable of and have been confirmed of the complete disintegration of the virion conformation. In other words, the innovative chemical substances of the present invention kill viruses and at least reduce the contagiousness of bacteria.

The following examples outline the chemical formula of the preferred embodiments of the substances of the present invention. Each of them represents an embodiment of the innovative chemical substances according to the present invention.

### EXAMPLE 1

A preferred embodiment of the chemical substance in accordance with the disclosure of the present invention has a chemical composition comprising an elongated structure such as in the following chemical formula:
Y—(CH₂)ₙ—COOH
where n is preferably an integer from 5 to 10.

The substance has a hydrocarbon long chain of hydrophobic nature and comprises a hydrophilic carboxyl group —COOH at one end and another functional group, generally represented as group Y, at the other. The chain of hydrocarbon may comprise a methylene —(CH₂)ₙ The end-bonded functional group Y is suitable for inflicting linkage of the molecule of the innovative substance onto an applicable material in a secured bonding manner. Preferred functional groups Y for inflicting this linking for the substance include hydroxyl —OH, mercapto —SH, amino —NH₂ and carboxyl —COOH groups. Examples of substances conforming to this chemical composition include at least the following:
(n+1)-hydroxy-alkanoic acid;
1, (n+1)-alkanedioic acid;
(n+1)-amino-alkanoic acid; and
(n+1)-mercapto-alkanoic acid.

Fabrication of these substances, HO(CH₂)ₙCOOH for example, can be achieved in a process generally exemplified as follows utilizing small amounts of raw material chemicals. Note that the following-described process serves only to demonstrate how the substance can be fabricated.

In approximately 45 ml of water, dissolve 10 grams of n-bromo alkanoic acid (Br(CH₂)ₙCOOH) and 5.4 grams of lithium hydroxide LiOH into an aqueous solution containing 5 ml of 1,4-Dioxane. The solution is then refluxed for approximately 10 hours and left cooled. 2N HCl is then added when the solution is subsequently cooled to room temperature for acidifying the solution to a pH value of approximately 4˜5. Ethyl acetate is then used to perform an extraction and the extraction layer is then washed utilizing both deionized water and saturated NaCl solution (brine). The extraction layer is then dried utilizing anhydrous magnesium sulfate in order to remove any water residue. Finally the extraction layer can further be dried in vacuum to obtain the (n+1)-hydroxylalkanoic acid compound HO(CH₂)ₙCOOH. This fabrication process is able to achieve a yield rate of over 90 percent.

### EXAMPLE 2

Chemical formula of another embodiment of the substance of the present invention also having a chemical composition comprising an elongated structure is 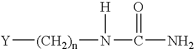
wherein n is preferably an integer from 4 to 10.

The substance has a long chain of hydrocarbon, a methylene for example, of hydrophobic nature, having a hydrophilic ureido group —N(C=O)NH₂ at one end and a functional group, Y, at the other. The functional group Y is suitable for implementing linkage of the substance molecule to materials of selected disinfection application. Preferred functional groups Y for inflicting this linking for the substance include methyl —CH₃, hydroxyl —OH, mercapto —SH, amino —NH₂ and carboxyl —COOH groups. Examples of substances conforming to this structural composition include at least the following:
(n-hydroxy-alkyl)-urea;
(n-mercapto-alkyl)-urea;
(n-amino-alkyl)-urea; and
(n+1)-uredio-alkanoic-urea.

### EXAMPLE 3

Formula of another embodiment of the substance of the present invention is: 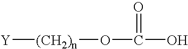
wherein n is an integer from 4 to 10.

Chemical composition of the substance is substantially an elongated structure, which is a long chain of hydrocarbon (methylene —(CH₂)ₙ) of hydrophobic nature, having a hydrophilic carbonate group —O(C═O)OH at one end and a functional group Y at the other for implementing linkage to materials of selected disinfection application. Suitable functional groups Y for inflicting this linking include methyl —CH₃, hydroxyl —OH, mercapto —SH, amino —NH₂ and carboxyl —COOH groups. Examples of substances conforming to this structural composition include at least the following:
carbonic acid monoalkyl ester;
carbonic acid mono-(n-hydroxy-alkyl) ester;
carbonic acid (n-mercapto-alkyl) ester;
carbonic acid (n-amino-alkyl) ester; and
(n+1)-carboxyoy-alkanoic acid.

### EXAMPLE 4

Another embodiment of the substance of the present invention is: 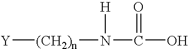
wherein n is an integer from 4 to 10.

The substance has a chemical composition comprising an elongated structure. Long chain of the substance is a methylene —(CH₂)ₙ hydrocarbon of hydrophobic nature, having a hydrophilic carbamate group, —NH(C═O)OH at one end and a functional group Y at the other. The functional group Y is suitable for implementing linkage to materials of selected disinfection application. Preferred functional groups for inflicting this linking for the substance include methyl —CH₃, hydroxyl —OH, mercapto —SH, amino —NH₂ and carboxyl —COOH groups. Examples of substances conforming to this structural composition include at least the following:
alkyl-carbamic acid;
(n-hydroxy-alkyl)-carbamic acid;
(n-mercapto-alkyl)-carbamic acid;
(n-amino-alkyl)-carbamic acid; and
(n+1)-carboxyamino-alkanoic acid.

### EXAMPLE 5

Chemical formula of another embodiment of the innovative substance is: 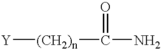
wherein n is an integer from 5 to 10.

The substance has a chemical composition comprising an elongated structure. It has a long chain of methylene hydrocarbon of hydrophobic nature, having a hydrophilic amido group —CONH₂ at one end and a functional group Y at the other. The functional group Y is suitable for implementing linkage to materials of selected disinfection application. Preferred functional group Y for inflicting this linking for the substance include methyl —CH₃, hydroxyl —OH, mercapto —SH, amino —NH₂ and carboxyl —COOH groups. Examples of substances conforming to this structural composition include at least the following:
(n+1)-hydroxy-alkanoic acid aminde;
(n+1)-mercapto-alkanoic acid aminde;
(n+1)-amino-alkanoic acid aminde; and
(n+1)-carbamoyl-alkanoic acid.

### EXAMPLE 6

Chemical formula of another embodiment of the innovative microorganism disinfecting substance is: 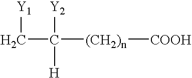
wherein n is preferably an integer from 3 to 9.

This substance has a chemical composition comprising an elongated structure that has a long chain of methylene hydrocarbon of hydrophobic nature, having a hydrophilic carboxyl group, —COOH at one end and two functional groups Y₁ and Y₂ at the other. Both the functional groups Y₁ and Y₂ are used for implementing linkage to materials of selected disinfection application. As compared to other embodiments described for the innovative substance, double functional groups achieve relatively enhanced linkage/bonding to the application material. Preferred functional groups Y₁ and Y₂ paired for inflicting this linking for the substance include methyl —CH₃, hydroxyl —OH and amino —NH₂ groups. Examples of substances conforming to this structural composition include at least the following:
(n+2)-methyl-alkanoic acid having hydrogen —H and methyl —CH₃ functional groups;
(n+2)-hydroxy-alkanoic acid having hydrogen —H and hydroxyl —OH functional groups;
(n+2)-amino-alkanoic acid having hydrogen —H and amino —NH₂ functional groups;
(n+2)-methyl-alkanoic acid having double methyl —CH₃ functional groups;
(n+2), (n+3)-dihydroxy-alkanoic acid having double hydroxyl —OH functional groups;
(n+2), (n+3)-diamino-alkanoic acid having double amino —NH₂ functional groups;
(n+2)-hydroxy-alkanoic acid having methyl —CH₃ and hydroxyl —OH functional groups;
(n+2)-amino-alkanoic acid having methyl —CH₃ and amino —NH₂ functional groups;
(n+3)-hydroxy-(n+2)-methyl-alkanoic acid having hydroxyl —OH and methyl—CH₃ functional groups;
(n+3)-hydroxy-(n+2)-amino-alkanoic acid having hydroxyl —OH and amino—NH₂ functional groups;
(n+3)-amino-(n+2)-methyl-alkanoic acid having amino —NH₂ and methyl —CH₃ functional groups; and
(n+3)-amino-(n+2)-hydroxy-alkanoic acid having amino —NH₂ and hydroxyl —OH functional groups.

Note here that although two functional groups Y₁ and Y₂ are exemplified here for implementing the linkage of the substance to an application surface, it is comprehensible for those skilled in the art that more than two functional groups serving the same purpose are possible.

### EXAMPLE 7

Chemical formula of another embodiment of the innovative microorganism disinfecting substance is: 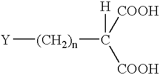
wherein n is preferably an integer from 4 to 10.

The substance has a chemical composition comprising an elongated structure that has a long chain of methylene hydrocarbon of hydrophobic nature, having two hydrophilic carboxyl groups —COOH at one end and another functional group Y at the other. The two carboxyl functional groups serve to enhance the substance molecules' binding to target microbes to be disinfected. Preferred functional groups Y for inflicting linking for the substance to the application material include methyl —CH₃, hydroxyl —OH, mercapto —SH, amino —NH₂ and carboxyl —COOH groups. Examples of substances conforming to this structural composition include at least the following:
2-alkyl-malonic acid;
2-(n-hydroxy-alkyl)-malonic acid;
2-(n-mercapto-alkyl)-malonic acid;
2-(n-amino-alkyl)- malonic acid; and
2-carboxy-alkanedioic acid.

Note here that although two hydrophilic carboxyl functional groups —COOH are used here for implementing improved adsorption of the substance to target microbes, it is comprehensible that more than two hydrophilic functional groups serving the same purpose are possible.

### EXAMPLE 8

Chemical formula of another embodiment of the innovative microorganism disinfecting substance is: 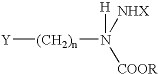
wherein n is preferably an integer from 4 to 10.

The substance has a chemical composition comprising an elongated structure that has a long chain of methylene hydrocarbon of hydrophobic nature. The long chain has two hydrophilic groups X and R bonded to itself via an amine —NH and a carboxylate —COO respectively. These two functional groups X and R, similar as in the substance described in Example 7, also serve to enhance the substance molecules' adsorption to target microbes to be disinfected. Preferred functional groups X include hydrogen —H, acetyl —Ac, chloro —Cl and bromo —Br groups. Preferred functional groups for R include hydrogen —H and methyl —CH₃ groups.

This substance also has a functional group Y at the opposite end of its long chain for application material linking. Preferred functional groups Y for inflicting this linking for the substance include methyl —CH₃, hydroxyl —OH, mercapto —SH, amino —NH₂ and carboxyl —COOH groups. Examples of substances conforming to this structural composition include at least the following:
Substances with hydrogen —H group selected for both its functional groups X and R, and methyl —CH₃, hydroxyl —OH, mercapto —SH, amino —NH₂ and carboxyl —COOH groups for group Y:
2-amino-alkanoic acid;
2-amino-(n+2)-hydroxy-alkanoic acid;
2-amino-(n+2)-mercapto-alkanoic acid;
2, (n+2)-diamino-alkanoic acid; and
2-amino-alkanedioic acid;
Substances with hydrogen —H and methyl —CH₃ groups selected respectively for its functional groups X and R, and methyl —CH₃, hydroxyl —OH, mercapto —SH, amino —NH₂ and carboxyl —COOH groups for group Y:
2-amino-alkanoic acid methyl ester;
2-amino-(n+2)-hydroxy-alkanoic acid methyl ester;
2-amino-(n+2)-mercapto-alkanoic acid methyl ester;
2, (n+2)-diamino-alkanoic acid methyl ester; and
2-amino-alkanedioic acid 1-methyl ester;
Substances with acetyl —Ac and hydrogen —H groups selected respectively for its functional groups X and R, and methyl —CH₃, hydroxyl —OH, mercapto —SH, amino —NH₂ and carboxyl —COOH for group Y:
2-acetylamino-alkanoic acid;
2-acetylamino-(n+2)-hydroxy-alkanoic acid;
2-acetylamino-(n+2)-mercapto-alkanoic acid;
2-acetylamino-(n+2)-amino-alkanoic acid; and
2-acetylamino-alkanedioic acid;
Substances with acetyl —Ac and methyl —CH₃ groups selected respectively for its functional groups X and R, and methyl —CH₃, hydroxyl —OH, mercapto —SH, amino —NH₂ and carboxyl —COOH for group Y:
2-acetylamino-alkanoic acid methyl ester;
2-acetylamino-(n+2)-hydroxy-alkanoic acid methyl ester;
2-acetylamino-(n+2)-mercapto-alkanoic acid methyl ester;
2-acetylamino-(n+2)-amino-alkanoic acid methyl ester; and
2-acetylamino-alkanedioic acid 1-methyl ester;
Substances with chloro —Cl and methyl —CH₃ groups selected respectively for its functional groups X and R, and methyl —CH₃, hydroxyl —OH, mercapto —SH, amino —NH₂ and carboxyl —COOH for group Y:
2-ammonium chloride-alkanoic acid methyl ester;
2-ammonium chloride-(n+2)-hydroxy-alkanoic acid methyl ester;
2-ammonium chloride-(n+2)-mercapto-alkanoic acid methyl ester;
2-ammonium chloride-(n+2)-amino-alkanoic acid methyl ester; and
2-ammonium chloride-alkanedioic acid 1-methyl ester;
Substances with bromo —Br and methyl —CH₃ groups selected respectively for its functional groups X and R, and methyl —CH₃, hydroxyl —OH, mercapto —SH, amino —NH₂ and carboxyl —COOH for group Y:
2-ammonium bromide-alkanoic acid methyl ester;
2-ammonium bromide-(n+2)-hydroxy-alkanoic acid methyl ester;
2-ammonium bromide-(n+2)-mercapto-alkanoic acid methyl ester;
2-ammonium bromide-(n+2)-amino-alkanoic acid methyl ester; and
2-ammonium bromide-alkanedioic acid 1-methyl ester;
Substances with chloro —Cl and hydrogen —H groups selected respectively for its functional groups X and R, and methyl —CH₃, hydroxyl —OH, mercapto —SH, amino —NH₂ and carboxyl —COOH for group Y:
2-ammonium chloride-alkanoic acid;
2-ammonium chloride-(n+2)-hydroxy-alkanoic acid;
2-ammonium chloride-(n+2)-mercapto-alkanoic acid;
2-ammonium chloride-(n+2)-amino-alkanoic acid; and
2-ammonium chloride-alkanedioic acid;
Substances with bromo —Br and hydrogen —H groups selected respectively for its functional groups X and R, and methyl —CH₃, hydroxyl —OH, mercapto —SH, amino —NH₂ and carboxyl —COOH for group Y:
2-ammonium bromide-alkanoic acid;
2-ammonium bromide-(n+2)-hydroxy-alkanoic acid;
2-ammonium bromide-(n+2)-mercapto-alkanoic acid;
2-ammonium bromide-(n+2)-amino-alkanoic acid; and
2-ammonium bromide-alkanedioic acid.

### EXAMPLE 9

Chemical formula of another embodiment of the innovative microorganism disinfecting substance is: 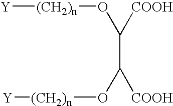
wherein n is preferably an integer from 4 to 10.

The substance has a chemical composition comprising double elongated structures each having a long chain of methylene hydrocarbon of hydrophobic nature. Each of the long chains can be considered to have its own hydrophilic group, carboxyl —C═OO as exemplified, bonded to its chain via a corresponding oxygen atom formed from the tartaric acid substance in an etherification reaction.

The multiple hydrophilic carboxyl functional groups —COOH, similar as in the substances described in Examples 7 and 8 respectively, serve the similar purpose of enhancing the substance molecules' adsorption to target microbes to be disinfected.

Each of the long chains of the substance also has a functional group Y at the opposite end of the hydrophilic carboxyl —COOH functional groups for implementing application material linking. Preferred functional groups for Y for inflicting this linking include methyl —CH₃, hydroxyl —OH, mercapto —SH, amino —NH₂ and carboxyl —COOH groups. Examples of substances conforming to this structural composition include at least the following:
2,3-bis-alkyloxy-succinic acid;
2,3-bis-(n-hydroxy-alkyloxy)-succinic acid;
2,3-bis-(n-mercapto-alkyloxy)-succinic acid;
2,3-bis-(n-amino-alkyloxy)-succinic acid; and
2,3-bis-(n-carboxyalkyloxy)-succinic acid.

### EXAMPLE 10

Formula of another embodiment of the innovative microorganism disinfecting substances with double long chains is: 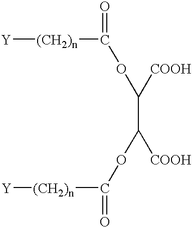
wherein n is preferably an integer from 3 to 9.

Likewise, the substance has a chemical composition comprising double elongated structures each having a long chain of methylene hydrocarbon of hydrophobic nature. Each of the long chains can be considered to have its own hydrophilic carboxyl —COOH group, which, again, serves to enhance the substance molecules' adsorption to target microbes to be disinfected. Unlike in the case of Example 9, however, each of the carboxyl groups is bonded to its corresponding methylene hydrocarbon long chain via a carboxylate —C═OO, formed in an esterification reaction utilizing alcohol and carboxylic acid.

For this substance, each of the long chains has a functional group Y at the end of its long chain opposite to the hydrophilic carboxyl functional group. The functional group Y is for application material linking. Preferred functional groups for Y for inflicting this linking for the substance include methyl —CH₃, hydroxyl —OH, mercapto —SH, amino —NH₂ and carboxyl —COOH groups. Examples of substances conforming to this structural composition include at least the following:
2,3-bis-alkanoyloxy-succinic acid;
2,3-bis-(n-hydroxy-alkanoyloxy)-succinic acid;
2,3-bis-(n-mercapto-alkanoyloxy)-succinic acid;
2,3-bis-(n-amino-alkanoyloxy)-succinic acid; and
2,3-bis-(n-carboxyalkanoyloxy)-succinic acid.

### EXAMPLE 11

Chemical formula of yet another embodiment of the innovative microorganism disinfecting substance with double long chains is: 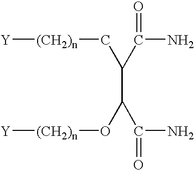
wherein n is preferably an integer from 4 to 10.

The substance has a chemical composition comprising double elongated structures each having a long chain of methylene hydrocarbon of hydrophobic nature. In a perspective, each of the long chains has its own hydrophilic amido —(C═O)NH₂ groups respectively bonded to itself. The multiple hydrophilic amido functional groups improve solubility and serve to enhance the substance molecules' adsorption to target microbes to be disinfected.

For the substance, each of the long chains has a functional group Y at the end of its long chain opposite to the hydrophilic amido functional groups for application material linking. Preferred functional groups for Y for inflicting this linking for the substance include methyl —CH₃, hydroxyl —OH, mercapto —SH, amino —NH₂ and carboxyl —COOH groups. Examples of substances conforming to this structural composition include at least the following:
2,3-bis-alkyloxy-succinamide;
2,3-bis-(n-hydroxy-alkyloxyysuccinamide;
2,3-bis-(n-mercapto-alkyloxy)succinamide;
2,3-bis-(n-amino-alkyloxy)succinamide; and
2,3-bis-(n-carboxyalkyloxy)-succinamide.

### EXAMPLE 12

Chemical formula of another embodiment of the innovative microorganism disinfecting substance with double long chains is: 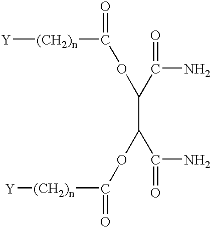
wherein n is preferably an integer from 3 to 9.

The substance also has a chemical composition comprising double elongated structures each having a long chain of methylene hydrocarbon of hydrophobic nature. Each of the long chains, in a perspective, has its own hydrophilic amido —CONH₂ functional group respectively bonded to itself via a chemical structure, the common C—O bond formed in an esterification reaction. They serve to enhance the substance molecules' adsorption to target microbes to be disinfected. Effectively, the hydrophilic amido groups are bonded to their respective hydrocarbon long chains via a corresponding carboxylate —COO.

For the substance, each of the long chains has a functional group Y at the end of its long chain opposite to the hydrophilic amido functional groups for application material linking. Preferred functional groups for Y for inflicting this linking for the substance include methyl —CH₃, hydroxyl —OH, mercapto —SH, amino —NH₂ and carboxyl —COOH groups. Examples of substances conforming to this structural composition include at least the following:
alkanoic acid 1,2-dicarbamoyl-2-alkanoyloxy-ethyl ester;
n-hydroxy-alkanoic acid 1,2-dicarbamoyl-2-(n-hydroxy-alkanoyloxy)-ethyl ester;
n-hydroxy-alkanoic acid 1,2-dicarbamoyl-2-(n-mercapto-alkanoyloxy)-ethyl ester;
n-amino-alkanoic acid 2-(n-amino-alkanoyloxy)-1,2-dicarbamoyl-ethyl ester; and
alkanedioic acid mono-[1,2-dicarbamoyl-2(n-carboxy-alkanoyloxy)-ethyl] ester.

Note that each of the preferred embodiments of the innovative substance exemplified and described in Examples 9˜12 above has a double-chain chemical composition. However, it suffices to indicate that it is possible to have more than two substantially parallel long chains as exemplified herein, both for enhanced aggregation in the process of adsorption to target microbes and enhanced linkage to the corresponding application material.

### EXAMPLE 13

Another preferred embodiment of the chemical substance in accordance with the disclosure of the present invention has a chemical composition comprising an elongated structure:
Y—(CH₂)ₙ—SO₃H
wherein n is preferably an integer from 6 to 12.

The substance has a long chain of methylene hydrocarbon of hydrophobic nature. The substance comprises a hydrophilic sulfonic acid (—SO₃H) group at one end and another functional group, Y, at the other. The end-attached functional group Y is suitable for inflicting linking or adsorbing of the substance itself onto the surface of an applicable material in a fixed manner. Preferred functional groups for Y for inflicting this linking/adsorption for the substance include hydroxyl —OH, mercapto —SH, amino —NH₂ and carboxyl —COOH groups. Examples of substances conforming to this structural composition include at least the following:
alkylsulfonic acid;
n-hydroxy-alkylsulfonic acid;
n-alkylsulfonic acid;
n-amino-alkylsulfonic acid; and
n-carboxy-alkylsulfonic acid.

Being effective in disinfection against various microbes including virus and bacteria, the obvious use of the chemical substance disclosed by the present invention is via application of the inventive substances directly to the target microbes. Typical of this direct application of the inventive disinfecting substance to the target microorganisms is to carry the substance molecules in a solution, normally aqueous, so that the solution may be applied, for example, via spraying, to the locations where the microbes are.

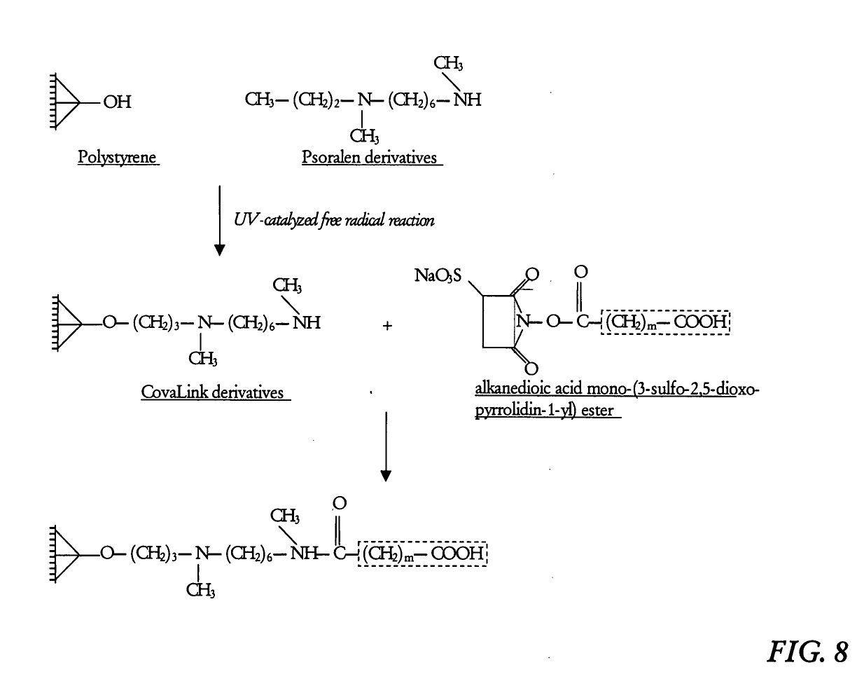

However, it is desirable to have the inventive substances deployed to the surface of, for example, house appliances or linked to the fibers of textiles such as that for respiratory masks. The optional linker functional groups described above bonded to the hydrocarbon long chain of the chemical composition of the inventive substance serve just this purpose. Different chemical functional groups can be selected for suitable linkage of the substance to different application surfaces. The chemical reaction schematically outlined in FIG. 8 shows how the hydrocarbon long chain of the molecules of the inventive substance can be linked to the surface of a polystyrene-based plastic material.

Surface of conventional polystyrene plastic material has no ready and appropriate functional group to implement this linkage by molecules of the chemical substance. Special treatment applied to the surface of the polystyrene material becomes necessary. The chemical reaction described in FIG. 8 demonstrates how psoralen derivatives applied to the surface of polystyrene plastic material is irradiated by an ultraviolet irradiation. This is an UV-catalyzed free radical reaction that produces ethereal bonds over the surface of the polystyrene plastic material. The polystyrene material having surface amine functional group is then provided for reaction with alkanedioic acid mono-(3-sulfo-2,5-dioxo-pyrrolidin-1-yl) ester. The result, as is shown in the chemical reaction of FIG. 8, is the linkage of the innovative substance to the surface of the polystyrene material. Such a linkage is a strong and secure covalent bonding of the inventive substance to the surface of the plastic material, which becomes a microorganism-disinfecting surface.

On the other hand, it is also possible to coat the surface of the polystyrene material with a functional polymer followed by the linkage of a chemical substance of the present invention to this coated surface. The result is another microorganism-disinfecting surface. Though, since the chemical substance molecules of the present invention are not directly linked to the surface of the polystyrene plastic material, the disinfection functionality is not as sustainable as when the substance molecules are directly bonded to the material surface. As the coated functional polymeric material is gradually lost, so is the disinfection capability becoming less effective.

Based on the processing described above, surface of the plastic portions of appliances and textiles can be made microbe-disinfecting. Products such as disinfecting mobile phone handsets, refrigerator door handles, computer keyboards as well as protective suits and masks are thus possible.

### Disinfection Efficacy Experiment Results

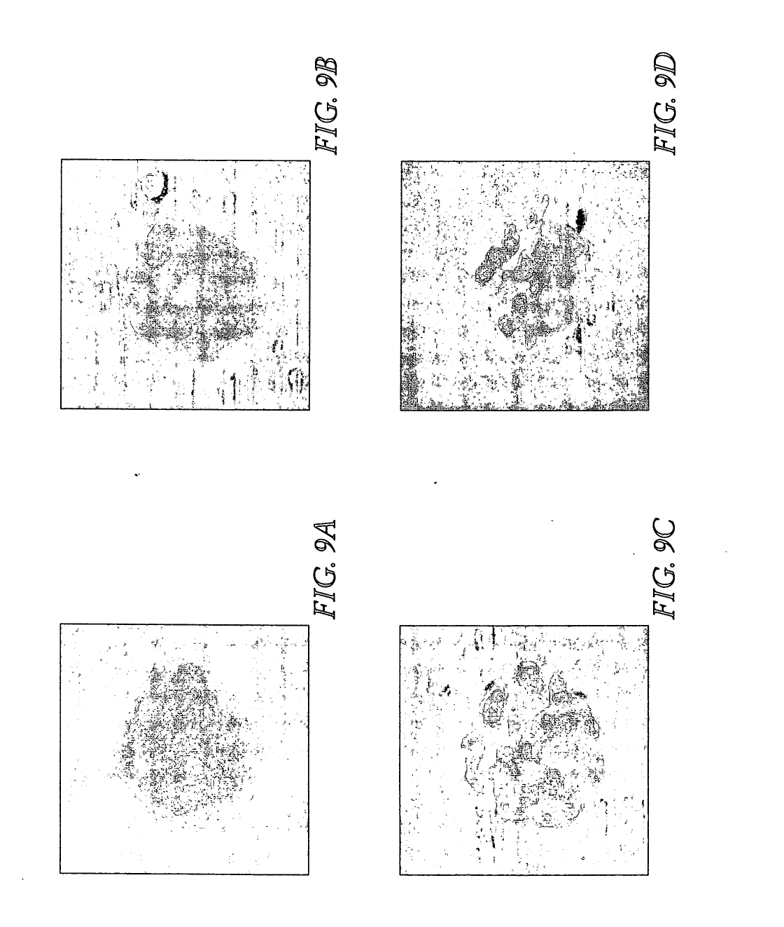

As a preferred embodiment of the innovative substance of the present invention, samples of the 8-hydroxyoctanoic acid were used in experimental treatments of coronavirus such as the SARS virus, enterovirus, and the Serratia bacterium. Treatment results were summarized respectively in FIGS. 9A˜9D, 10A and 10B, and 11A and 11B of the drawing.

FIGS. 9A˜9D respectively are atomic force microscope (AFM) pictures of a SARS virion taken at different stages of treatment by the innovative 8-hydroxyoctanoic acid organic substance of the present invention. The virion in pictured in FIG. 9A is before treatment by the substance, and the picture shows a generally intact microbial conformation, with the coronavirus' characteristic spikes identifiably visible.

The AFM picture in FIG. 9B is the same virion of FIG. 9A a few minutes after treatment by the substance. At this stage, the conformation of the coronavirus has seen disruption, with at least one spike component seen disintegrated from the virion itself.

Then, at FIG. 9C, a few more minutes into treatment, the virion is observed to become collapsed further more, and in FIG. 9D, the virion can be considered completely killed as its biological components are seen scattered around instead as compared to the picture of FIG. 9A.

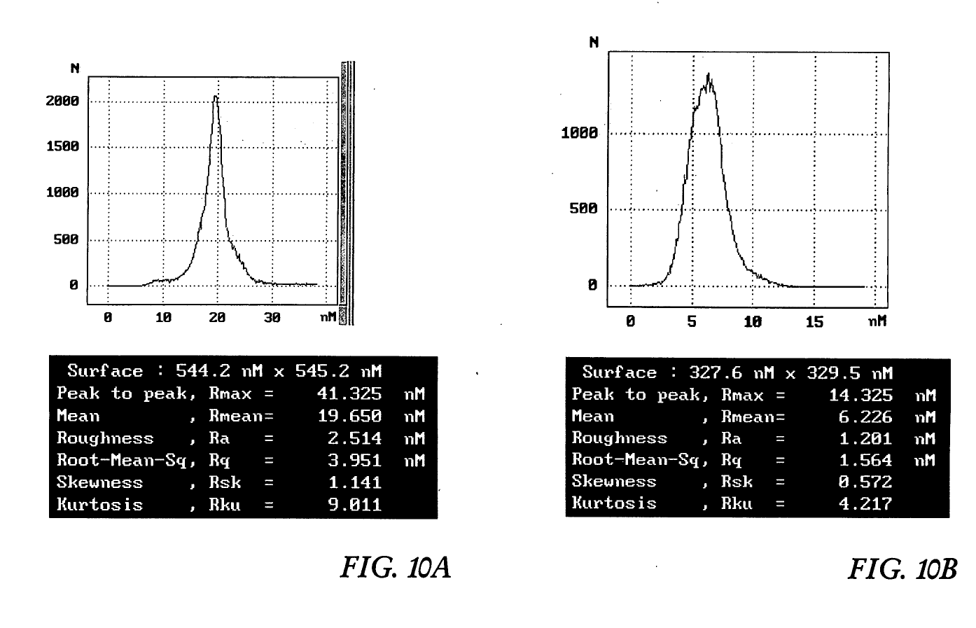

FIGS. 10A and 10B respectively are the AFM height images of an enterovirus virion before and after the treatment by the inventive substance 8-hydroxyoctanoic acid. The pre- and post-treatment AFM height images confirm the collapse of the conformation of the virion. There was a height reduction from about 2,000 to about 1,300 nm after the treatment by the 8-hydroxyoctanoic acid. This represents a significant collapse of the protein envelope of the enterovirus virion due to the substance treatment.

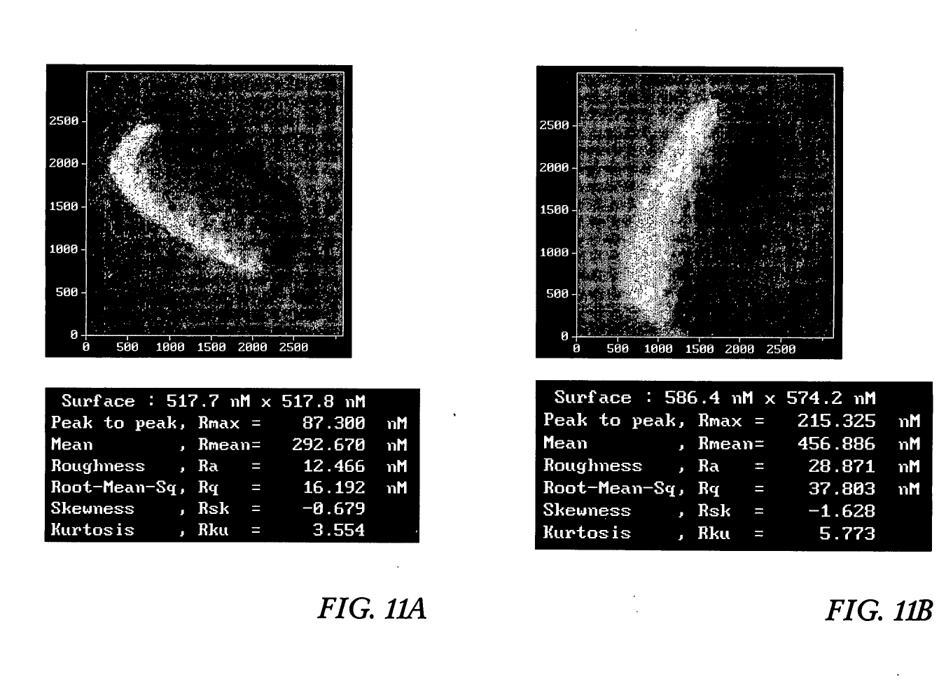

FIGS. 11A and 1B respectively are the AFM pictures of a bacterium of the Serratia species taken before and after the treatment by the 8-hydroxyoctanoic acid organic substance of the present invention. In addition to the comparative pictures, experimental data listed in FIGS. 11A and 1B respectively show that the bacterium suffered conformation damages as suggested by the increased surface roughness measured by an AFM.

While the above is a full description of the specific embodiments, various modifications, alternative constructions and equivalents may be used without departing from the spirit and scope of the invention. Therefore, the above description and illustrations should not be taken as limiting the scope of the present invention which is defined by the appended claims.

1. A substance for breaking down the conformation of a microbe into biological components, said substance having a chemical composition of hydrophobic nature having bonded to at least one structural point thereof a hydrophilic functional group.

2. The substance of claim 1 wherein said chemical composition comprises a long chain of hydrocarbon.

3. The substance of claim 1 wherein said at least one structural point of said chemical composition is end point of said chemical composition.

4. The substance of claim 1 wherein said chemical composition further having bonded to at least another structural point thereof a functional group for affixing said substance to an application surface where said microbe is located.

5. The substance of claim 4 wherein said at least another structural point of said chemical composition is opposite to said at least one structural point where said hydrophilic functional group is bonded.

6. A substance for breaking down the conformation of a microbe into biological components, said substance having a chemical composition of hydrophobic nature comprising a long chain of hydrocarbon and having bonded to one end of said long chain at least one hydrophilic functional group.

7. The substance of claim 6 wherein said chemical composition further having bonded to the end of said long chain opposite to the end of said at least one hydrophilic functional group at least a functional group for affixing said substance to an application surface where said microbe is located.

8. A substance for breaking down the conformation of a microbe into biological components, said substance having a chemical composition of hydrophobic nature having bonded to at least one structural point thereof a hydrophilic functional group, wherein a multiplicity of molecules of said substance being adsorbed by said hydrophilicity to the microbial surface of said microbe and assembled gradually into aggregates, said aggregates at close proximity of said microbial surface exerting adsorptive interaction forces to nearby ones of said microbe biological components competing with the absorptive interaction forces among said nearby biological components, and said competition disrupting said conformation by breaking out of structural binding equilibrium among said biological components and thereby breaking down and denaturing said microbe.

9. The substance of claim 8 wherein said chemical composition comprises a long chain of hydrocarbon.

10. The substance of claim 8 wherein said at least one structural point of said chemical composition is end point of said chemical composition.

11. The substance of claim 8 wherein said chemical composition further having bonded to at least another structural point thereof a functional group for affixing said substance to an application surface where said microbe is located.

12. The substance of claim 11 wherein said at least another structural point of said chemical composition is opposite to said at least one structural point where said hydrophilic functional group is bonded.

13. A substance for breaking down the conformation of a microbe into biological components, said substance having a chemical composition of hydrophobic nature comprising a long chain of hydrocarbon and having bonded to one end of said long chain at least one hydrophilic functional group, wherein a multiplicity of molecules of said substance being adsorbed by said hydrophilicity to the microbial surface of said microbe and assembled gradually into aggregates, said aggregates at close proximity of said microbial surface exerting adsorptive interaction forces to nearby ones of said microbe biological components competing with the absorptive interaction forces among said nearby biological components, and said competition disrupting said conformation by breaking out of structural binding equilibrium among said biological components and thereby breaking down and denaturing said microbe.

14. The substance of claim 13 wherein said chemical composition further having bonded to the end of said long chain opposite to the end of said at least one hydrophilic functional group at least a functional group for affixing said substance to an application surface where said microbe is located.

15. A substance for breaking down the conformation of a microbe into biological components, said substance having a chemical composition of hydrophobic nature comprising a long hydrocarbon chain and having bonded to one end of said long chain at least one hydrophilic carboxyl group —COOH.

16. The substance of claim 15 wherein said chemical composition further having bonded to the end of said long chain opposite to the end of said at least one hydrophilic carboxyl group —COOH at least a functional group for affixing said substance to an application surface where said microbe is located.

17. The substance of claim 16 wherein said at least one functional group for affixation is a hydroxyl group —OH or a derivative thereof.

18. The substance of claim 16 wherein said at least one functional group for affixation is a mercapto group —SH or a derivative thereof.

19. The substance of claim 16 wherein said at least one functional group for affixation is an amino group —NH₂ or a derivative thereof.

20. The substance of claim 16 wherein said at least one functional group for affixation is a carboxyl group —COOH or a derivative thereof.

21. A substance for breaking down the conformation of a microbe into biological components, said substance having a chemical composition of hydrophobic nature comprising a long hydrocarbon chain and having bonded to one end of said long chain at least one ureido group —N(C═O)NH₂.

22. The substance of claim 21 wherein said chemical composition further having bonded to the end of said long chain opposite to the end of said at least one hydrophilic carboxyl group —COOH at least a functional group for affixing said substance to an application surface where said microbe is located.

23. The substance of claim 21 wherein said at least one functional group for affixation is a methyl group —CH₃ or a derivative thereof.

24. The substance of claim 21 wherein said at least one functional group for affixation is a hydroxyl group —OH or a derivative thereof.

25. The substance of claim 21 wherein said at least one functional group for affixation is a mercapto group —SH or a derivative thereof.

26. The substance of claim 21 wherein said at least one functional group for affixation is an amino group —NH₂ or a derivative thereof.

27. The substance of claim 21 wherein said at least one functional group for affixation is a carboxyl group —COOH or a derivative thereof.
